(19) United States
(12) Patent Application Publication
(10) Pub. No.: US 2026/0088161 A1
(43) Pub. Date: Mar. 26, 2026

(54) METHOD, APPARATUS, AND COMPUTER PROGRAM PRODUCT FOR GENERATING PREDICTED CARE COORDINATION DATA OBJECTS

(71) Applicant: McKesson Corporation, Irving, TX (US)

(72) Inventors: Judith Payne, New Caney, TX (US); Jody Sheehan Garey, Houston, TX (US)

(21) Appl. No.: 19/341,253

(22) Filed: Sep. 26, 2025

Related U.S. Application Data
(60) Provisional application No. 63/699,494, filed on Sep. 26, 2024.

(51) Int. Cl. G16H 40/20; G16H 10/60

(52) U.S. Cl. CPC G16H 40/20; G16H 10/60

(57) ABSTRACT

A method, apparatus, and computer program product are provided for generating predicted care coordination data objects using a machine learning-based predictive model. The model is trained to analyze complex, high-dimensional electronic health record (EHR) data to automatically estimate patient acuity levels, skilled care durations, and other care coordination parameters for scheduled appointments. These predictions are used to dynamically and prospectively update scheduling and resource allocation systems, improving operational efficiency and reducing manual workload. Unlike conventional systems or human-based methods, the disclosed system continuously adapts to evolving clinical data and care patterns, enabling real-time, data-driven decision-making. The integration of the predictive model with scheduling and allocation systems allows for automated adjustments to appointment durations, staffing levels, and resource needs, thereby enhancing care delivery and staff productivity. The system provides a scalable approach to acuity estimation thereby improving scheduling and resource allocation systems.

### CROSS REFERENCE TO RELATED APPLICATION

This application claims priority to U.S. Application No. 63/699,494, filed Sep. 26, 2024, the entire contents of which is hereby incorporated by reference.

### TECHNOLOGICAL FIELD

Embodiments of the present disclosure relate generally to computing systems, and more particularly, to methods, apparatuses, and computer program products for generating predicted care coordination data objects.

### BACKGROUND

Predicting proper nurse staffing levels in infusion centers is critical for safe, timely, and quality care. However, determining adequate staffing can be challenging, as many variables impact staffing needs. Today's complex therapies require close monitoring for adverse reactions, but it can be difficult to foresee how much time each patient will require. High patient turnover can also complicate staffing estimates, as some treatments are brief while others last hours. Patients may also remain in the clinic longer than expected, potentially straining nursing resources. Universally accepted nurse-to-patient ratios do not exist for infusion centers. Staffing decisions typically rely on estimates that are based on patient acuity and treatments to be given, and require significant interaction with systems such as a scheduling system, resource allocation system, and/or the like.

In outpatient oncology and other clinical care environments, acuity serves as a critical measure of the complexity of patient care and an estimate of the nursing workload required to deliver that care. Accurately assessing acuity is essential for determining appropriate staffing levels, balancing infusion room schedules, improving nurse satisfaction and productivity, and ultimately reducing staff turnover.

Conventional acuity assessment methods often rely on manual input or subjective evaluation, which can inadvertently increase nursing workload and introduce variability across different practitioners and clinical settings. These methods are prone to inconsistencies due to differences in interpretation, documentation practices, and institutional protocols. Furthermore, there is often a poor understanding of the validity and reliability of acuity measures, which undermines confidence in their use for operational decision-making.

Many healthcare providers have technology-based infrastructure in place to handle or assist with scheduling and allocation of resources. However, existing systems present significant limitations that hinder their effectiveness and scalability.

The lack of standardization and automation in acuity estimation tools creates barriers to proactive staffing and resource planning. Without a reliable system to predict acuity prospectively, healthcare providers are forced to rely on reactive scheduling and allocation strategies, such as manually interacting with scheduling and allocation systems, which can lead to overscheduling, understaffing, and inefficient use of system resources.

### BRIEF SUMMARY

The Oncology Nursing Society (ONS) recommends an acuity-based staffing model for ambulatory oncology, but scheduling and resource allocation systems lack the ability to effectively control scheduling and resource allocation based on acuity. As the study of oncology and cancer treatment evolves, findings relating to the significance of certain data from a patient's electronic health record (EHR) in determining acuity levels could shift. Additionally, EHR data for a patient with a healthcare appointment can change in between the time an appointment is scheduled and when the appointment occurs.

Example embodiments provided herein provide a predictive care coordination model, which may be referred to as or include an acuity model. Accordingly, certain example embodiments automatically predict patient acuity, staffing needs, or a combination thereof and may provide one or more improvements to one or more other systems, such as, but not limited to, one or more scheduling systems and one or more allocation systems (e.g., a staffing system). In this regard, a prediction and other operations performed herein may be described as “automatic” due to program code stored on memory being executed in response to a condition being satisfied that is discernable to a computer. For example, an operation can occur automatically on a routine basis, or automatically in response to electronic data satisfying a condition. Example embodiments provided herein provide an acuity rating system that reflects the estimated nursing time needed for coordination, delivery, and documentation of care.

Example embodiments identify electronic health record (EHR) information and features for predicting acuity, the duration of skilled care needed for an appointment, and other parameters. Example embodiments provide and train a machine-learning model to predict an acuity level (which also may be referred to as an acuity score, and may reflect one or more of: (i) the complexity of nursing care needed for one or more patients; (ii) the nursing skill level needed to care for one or more patients; (iii) the time needed for one or more nurses to spend with one or more patients; (iv) the severity of one or more medical conditions of one or more patients; and (v) any combination of (i)-(iv)). The acuity level may be predicted or calculated for one or more patients with future scheduled appointments, or for appointments currently being scheduled for one or more patients, based on available EHR data. Given that a substantial amount of data can be captured in an EHR for one or more patients, implementing example embodiments using a machine learning model enables embodiments to efficiently identify trends or data points and their respective impacts to one or more acuity scores for one or more patients.

Certain example embodiments therefore prospectively determine acuity levels and staffing requirements in infusion therapy. By integrating real-time and historical patient data, including treatment complexity, comorbidities, and clinical risk factors, certain example embodiments deliver dynamic acuity scores and staffing recommendations that enable proactive, data-driven decisions.

Integrating the machine-learning model with a scheduling system and/or resource allocation system provides improvements to the scheduling system and/or resource allocation system, by triggering an automatic adjustment of scheduling data and/or resource allocation data based on a predicted care coordination data object. For example, according to example embodiments, the model of the present disclosure provides accurate predictions of one or more of: (i) the time needed for a patient appointment (e.g. the total amount of time that a patient may be present for an appointment or the total amount of nursing time that is needed to care for a patient during an appointment); (ii) resource allocation needed for or related to one or more appointments for one or more patients; (iii) staffing needs (e.g. number of staff needed to care for a patient in a given timeframe and/or number of staff needed to care for one or more patients over the course of one or more appointments or other timeframes); (iv) staffing skill level needed to deliver care to one or more patients; and (v) amounts and/or types of medical equipment, pharmaceuticals, and/or other medical supplies needed to deliver care to one or more patients over the course of one or more appointments or other designated timeframe(s).

The scheduling system is therefore improved by setting estimated durations for appointments and/or skilled care durations of appointments based on one or more predicted care coordination data objects. The scheduling system may automatically increase estimated duration and/or estimated skilled-care duration needed for initially-scheduled appointments based on one or more predicted care coordination data objects. Increasing an initially scheduled appointment duration and/or skilled care duration can improve the scheduling system by limiting or preventing overscheduling. The scheduling system can be further improved by decreasing estimated durations or decreasing estimated durations needed for initially scheduled appointment. Decreasing an initially scheduled appointment duration and/or skilled care duration can improve the scheduling system by causing the scheduling system to automatically add additional appointment availability for other patients on a certain day. The resource allocation system is therefore improved by automatically and accurately determining resource needs based on predicted care coordination data objects.

Example embodiments may be advantageously implemented within or integrated with systems associated with outpatient healthcare and may differ from systems implemented for in-patient or hospital use that may be used for staffing based on a number of patients or a number of predicted patients present at a given time. In contrast to systems utilized in in-patient environments, example embodiments can use real-time EHR data pertaining to patients scheduled for an appointment in the future, to impact scheduling data and resource allocation data. Example embodiments may determine one or more acuity levels, or acuity scores, at the individual visit level, for scheduled outpatient appointments, whereas acuity levels for in-patient systems may refer to a longer term or continually changing, or in-flux, needs for admitted hospital patients.

A system and/or apparatus is provided, comprising a memory and one or more processors communicatively coupled to the memory, the one or more processors configured to receive an indication of a clinical event indication data object, and access an input data object associated with the clinical event indication data object. The memory and the one or more processors are further configured to apply the input data object and the clinical event indication data object to a care coordination predictive model to generate a predicted care coordination data object, and cause transmission of one or more data elements of the predicted care coordination data object to at least one of a scheduling system or an allocation system, wherein at least one of scheduling data associated with the clinical event indication data object or allocation data associated with the clinical event indication data object is determined or modified is based on the predicted care coordination data object.

The memory and the one or more processors may be further configured to receive an indication of a change to the input data object associated with the clinical event indication data object, and, in response to receiving the indication of the change, apply the input data object and the clinical event indication data object to generate an updated predicted care coordination data object. The memory and the one or more processors may be further configured to cause transmission of one or more data elements of the predicted care coordination data object to at least one of a scheduling system or an allocation system, wherein at least one of scheduling data associated with the clinical event indication data object or allocation data associated with the clinical event indication data object is modified based on the predicted care coordination data object.

According to certain embodiments, the input data object comprises or is generated from an electronic health record (EHR) associated with the clinical event indication data object. The memory and the one or more processors are further configured to generate and cause display of, at a user device, a dashboard report including one or more data elements of the predicted care coordination data object to at least one of a scheduling system or an allocation system.

The one or more data elements of the predicted care coordination data object may comprise a predicted skilled care duration. The one or more data elements of the predicted care coordination data object may comprise an acuity level.

The memory and the one or more processors may be further configured to access an updated input data object associated with the clinical event indication data object, and apply the updated input data object to the care coordination predictive model to generate an updated predicted care coordination data object, wherein the at least one of the scheduling data or the allocation data is further updated based on the updated input data object.

According to certain embodiments, the memory and the one or more processors are further configured to access a scheduling system on a routine basis to generate updated predicted care coordination data objects, wherein the at least one of the scheduling data or the allocation data is further modified based on the updated predicted care coordination data objects.

A computer-implemented method is provided, comprising receiving an indication of a clinical event indication data object, and accessing an input data object associated with the clinical event indication data object. The computer-implemented method further comprises applying the input data object and the clinical event indication data object to a care coordination predictive model to generate a predicted care coordination data object, and causing transmission of one or more data elements of the predicted care coordination data object to at least one of a scheduling system or an allocation system, wherein at least one of scheduling data associated with the clinical event indication data object or allocation data associated with the clinical event indication data object is determined or modified is based on the predicted care coordination data object.

A computer program product is provided, comprising at least one non-transitory computer-readable storage medium having computer-executable program code instructions stored therein, the computer-executable program code instructions comprising program code instructions to receive an indication of a clinical event indication data object, and access an input data object associated with the clinical event indication data object.

The computer-executable program code instructions comprising program code instructions to apply the input data object and the clinical event indication data object to a care coordination predictive model to generate a predicted care coordination data object, and cause transmission of one or more data elements of the predicted care coordination data object to at least one of a scheduling system or an allocation system, wherein at least one of scheduling data associated with the clinical event indication data object or allocation data associated with the clinical event indication data object is determined or modified is based on the predicted care coordination data object.

The above summary is provided merely for purposes of summarizing some example embodiments of the disclosure so as to provide a basic understanding of some aspects of the disclosure. Accordingly, it will be appreciated that the above-described example embodiments are merely examples and should not be construed to narrow the scope or spirit of the disclosure in any way. The scope of the disclosure encompasses many potential embodiments, some of which will be further described below, in addition to those summarized herein.

### BRIEF DESCRIPTION OF THE SEVERAL VIEWS OF THE DRAWINGS

Having thus described embodiments of the disclosure in general terms, reference will now be made to the accompanying drawings, which are not necessarily drawn to scale, and wherein:

FIG. 1 is an example overview of a system that can be used to practice some example embodiments described herein;

FIG. 2 is an exemplary schematic diagram of an apparatus in accordance with some example embodiments; and

FIGS. 3 and 4 are flowcharts of operations that may be performed in accordance with some example embodiments.

FIGS. 5-11 are example user interfaces provided according to some example embodiments; and

### DETAILED DESCRIPTION

Some embodiments of the present disclosure will now be described more fully hereinafter with reference to the accompanying drawings, in which some, but not all embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout.

As used herein, where a computing device is described to receive data from another computing device, it will be appreciated that the data may be received directly from the other computing device and/or may be received indirectly via one or more intermediary computing devices, such as, for example, one or more servers, relays, routers, network access points, and/or the like. Similarly, where a computing device is described herein to transmit data to another computing device, it will be appreciated that the data may be sent directly to the other computing device or may be sent to the other computing device via one or more interlinking computing devices, such as, for example, one or more servers, relays, routers, network access points, and/or the like.

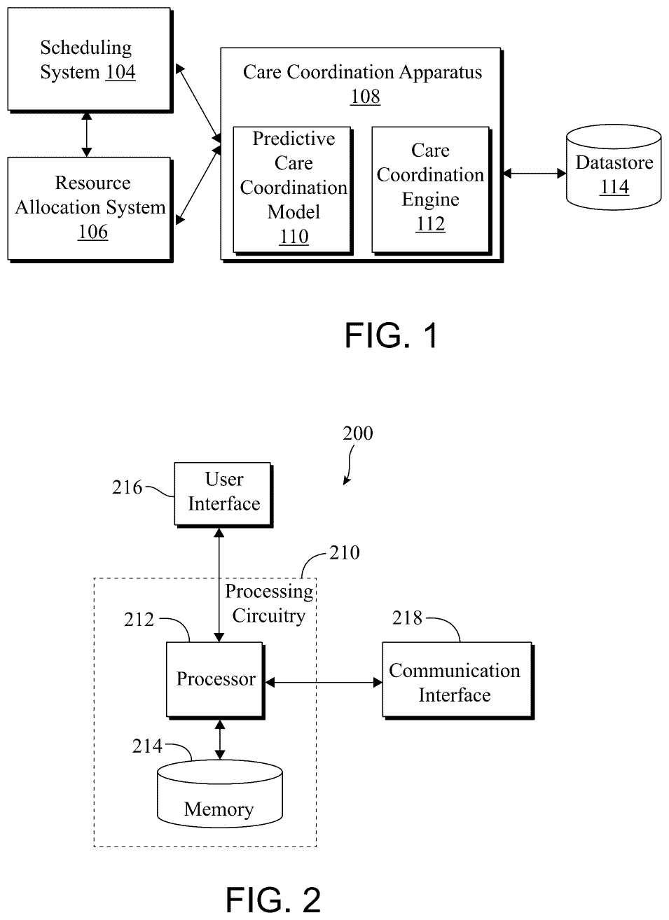

FIG. 1 is an overview of a system that can be used to practice certain example embodiments. The scheduling system 104 may be any processor-driven device that facilitates the scheduling of appointments on behalf of an entity, such as but not limited to a healthcare provider. The healthcare provider may include a clinic, an outpatient clinic, an outpatient oncology care clinic, a physician's office, or the like. The scheduling system 104 can be configured to manage appointments, facilitate booking of patient appointments such as via a website, mobile apps, internal applications such as those utilized by staff that scheduling appointment by phone, or an application programming interface (API) of another system, or the like. The scheduling system 104 can display real-time availability of the healthcare provider for scheduling. In this regard, if multiple users access the scheduling system, appointments scheduled by other users can be updated in an interface in real-time to prevent or reduce scheduling conflicts or overscheduling. The scheduling system 104 can be configured to track past and future appoints for patient and/or providers. The scheduling system 104 may be configured to maintain a waitlist for appointments, and to enable automatic notification to a patient when an appointment or earlier appointment that is potentially available for the patient becomes open, such as due to cancellation, rescheduling, or the like. The scheduling system can be implemented as a server, distributed system, or the like. According to certain embodiments provided herein, the resource allocation system 106 can perform resource allocations processes based on data received from the care coordination apparatus 108.

The scheduling system 104 may be integrated with a resource allocation system 106, configured to facilitate allocation of staff, equipment, supplies, appointment rooms, pharmaceuticals, or the like, and/or to manage clinic capacity for one or more physical locations of business of a healthcare provider. According to certain embodiments, the resource allocation system 106 uses information from the scheduling system 104 to allocate resources for the healthcare provider with regard to scheduled patient appointments. In this regarding, the resource allocation system 106 is configured to manage shifts for nurses, doctors, admixture technicians, pharmacy technicians, and other staff. According to certain embodiments, one organization may staff professionals in a local area but at multiple different sites in geographical proximity, and the resource allocation system 106 directs or diverts the allocation of such staff accordingly. The resource allocation system 106 may be configured to manage the allocation of rooms and medical equipment for specialized treatments and procedures. The resource allocation system 106 may include or may be integrated with (although not depicted in FIG. 1) a supply ordering system configured to order supplies and pharmaceuticals needed to provide services scheduled as reflected in the scheduling system 104. According to certain embodiments provided herein, the resource allocation system 106 can perform resource allocation processes based on data received from the care coordination apparatus 108.

The care coordination apparatus 108 is provided according to example embodiments to improve the scheduling system 104 and the resource allocation system 106. The care coordination apparatus 108 can be implemented as a server or a distributed system, and includes a predictive care coordination model 110, and care coordination engine 112.

The predictive care coordination model 110 is a machine learning model, including data representations of nodes (e.g., neural network nodes, decision tree nodes, Markov model nodes, other nodes, or combinations thereof) and connections between nodes (e.g., weighted or unweighted unidirectional or bidirectional connections). In certain embodiments, the predictive care coordination model 110 includes a representation of memory (e.g., providing long short-term memory functionality). The predictive care coordination model 110 is configured to generate a predicted care coordination data object, based on a clinical event indication data object (e.g., information pertaining to a scheduled appointment).

A predicted care coordination data object can include any data predicted by the predictive care coordination model 110 that could impact the patient and/or healthcare provider, such as information impacting scheduling, resource allocation and/or the like. The predicted care coordination data object can include an acuity level, a predicted appointment time, predicted staffing types and respective appointment durations and/or skilled care duration needed. Skilled care duration may differ from an appointment duration. An appointment duration may be longer than skilled care duration and may represent the duration a patient is expected to be at a healthcare provider. Skilled care duration is typically shorter than the appointment duration and includes a duration and/or estimated duration that a particular skilled worker, such as a nurse, is needed or predicted to be needed for an appointment. Skilled care duration may include the time a nurse spends with a patient during an infusion appointment. The predicted care coordination data object can include medication, supplies, rooms, other resources, and/or the like, predicted as needed for a scheduled appointment.

The predictive care coordination model 110 may be trained using a patient's EHR, such as from datastore 114, and corresponding labels. A label may include one or more data points, such as but not limited to data fields comprised by a predicted care coordination data object. A label may include known or true data associated with a clinical event indication data object. For example, a label may include an acuity level as determined by a nurse or other practitioner. A label can include actual appointment times logged by a system or staff member after completion of an appointment. A label can include indications of actual resources, such as supplies, medication, staff, equipment, or the like, noted as utilized for an appointment that has occurred. According to certain embodiments, some information pertaining to clinical care event indication data objects can be provided as labels on an ongoing basis to routinely improve the predictive care coordination model 110.

The predictive care coordination model 110 can be trained using a machine learning framework such that the model weights different features (e.g., data from an EHR) according to which data fields serve as predictors to the data comprised in a predicted care coordination data object, and to what extent the data fields are predictors. The predictive care coordination model 110 can then generate a predicted care coordination data object based on a clinical event indication data object.

The care coordination engine 112 may be a server and/or computing entity configured to direct the predictive care coordination model 110 to generate predicted care coordination data object. As an example, the care coordination engine 112 can be configured to trigger application of a clinical event indication data object for each scheduled appointment in a scheduling system 104, on a routine basis, such as daily, or the like. As another example, in response to detecting a change or addition to a patient's EHR, for a patient that is scheduled for an upcoming appointment in the scheduling system 104, the care coordination engine 112 may trigger the predictive care coordination model 110 to generate predicted care coordination data object. In this regard, the generated predicted care coordination data object can be a new instance of a predicted care coordination data object even if one was previously generated for a particular appointment instance (e.g., clinical event indication data object).

After a predicted care coordination data object is generated, the care coordination engine 112 causes transmission of one or more data elements of the predicted care coordination data object to at least one of the scheduling system 104 and/or the allocation system 106. For example, the care coordination engine 112 transmits, to the scheduling system 104, an updated appointment duration that differs from a currently scheduled duration time. As another example, the care coordination engine 112 transmits, to the allocation system 106, data pertaining to a staffing need, or other resource, that may differ from resources that are currently allocated for an appointment.

In this regard, the care coordination apparatus 108 may be implemented according to certain example embodiments such that no implementation changes, are needed at the scheduling system 104 and/or resource allocation system 106. The care coordination apparatus 108 can be implemented so as to integrated with an existing API of the scheduling system 104 and/or resource allocation system 106.

However, according to certain embodiments, the care coordination apparatus 108 can transmit the predicted care coordination data object and its contents, such that an existing scheduling system 104 and/or resource allocation system 106 unpacks and interprets the contents, and optionally performs updates to scheduling data and/or resource allocation data accordingly.

In any event, the scheduling system 104 and/or resource allocation system 106 are improved, due to the receipt of one or data elements of the predicted care coordination data object.

In certain example embodiments, the care coordination apparatus 108 may be configured as or may comprise a switch or router that evaluates, modifies, reformats, generates, and/or routes clinical event indication data objects. For example, given a clinical event indication data object that represents a scheduled appointment or appointment request, the care coordination engine 112 can generate one or more predicted care data coordination data object, and modify one or more data elements of the clinical care event indication data object, and route the modified clinical care event indication data object to the scheduling system 104 and/or resource allocation system 106.

The system of FIG. 1 may include other components not necessarily illustrated. For example, the system may include a user device configured to access and interact with any of the components. For example, a user device may enable a user to schedule or request an appointment by accessing the scheduling system 104, such that new scheduling data is created and generated. A user device may enable a user to access create, or update available resource information, or resource allocation data via the resource allocation system 106. A user device may enable a user to provide inputs, such as labels to be used to train the predictive care coordination model 110, via the care coordination apparatus 108. A user device may be used to display dashboards and/or reports generated according to example embodiments.

Referring now to FIG. 2, apparatus 200 is a computing device(s) configured for implementing scheduling system 104, resource allocation system 106, care coordination apparatus 108, predictive care coordination model 110, and/or care coordination engine 112, according to example embodiments.

Apparatus 200 may at least partially or wholly embody or be embodied by any of the scheduling system 104, resource allocation system 106, care coordination apparatus 108, predictive care coordination model 110, and/or care coordination engine 112. Apparatus 200 may therefore implement any of the components of FIG. 1 in accordance with some example embodiments, or may be implemented as a distributed system that includes any of the components of FIG. 1, and/or associated network(s).

It should be noted that the components, devices, and elements illustrated in and described with respect to FIG. 2 may not be mandatory and thus some may be omitted in certain embodiments. For example, FIG. 2 illustrates a user interface 216, as described in more detail below, which may be optional in certain components (such as when a component of FIG. 1 is implemented as a service communicatively connected to a workstation or other user device). Additionally, some embodiments may include further or different components, devices, or elements beyond those illustrated in and described with respect to FIG. 2.

Continuing with FIG. 2, processing circuitry 210 may be configured to perform actions in accordance with one or more example embodiments disclosed herein. In this regard, the processing circuitry 210 may be configured to perform and/or control performance of one or more functionalities of apparatus 200 in accordance with various example embodiments. The processing circuitry 210 may be configured to perform data processing, application execution, and/or other processing and management services according to one or more example embodiments. In some embodiments apparatus 200, or a portion(s) or component(s) thereof, such as the processing circuitry 210, may be embodied as or comprise a circuit chip. The circuit chip may constitute means for performing one or more operations for providing the functionalities described herein.

In some example embodiments, the processing circuitry 210 may include a processor 212, and in some embodiments, such as that illustrated in FIG. 2, may further include memory 214. The processing circuitry 210 may be in communication with or otherwise control a user interface 216, and/or a communication interface 218. As such, the processing circuitry 210, and/or apparatus 200 may be embodied as a circuit chip (e.g., an integrated circuit chip) configured (e.g., with hardware, software, or a combination of hardware and software) to perform operations described herein.

The processor 212 may be embodied in a number of different ways. For example, the processor 212 may be embodied as various processing means such as one or more of a microprocessor or other processing element, a coprocessor, a controller, or various other computing or processing devices including integrated circuits such as, for example, an ASIC (application specific integrated circuit), an FPGA (field programmable gate array), or the like. Although illustrated as a single processor, it will be appreciated that the processor 212 may comprise a plurality of processors. The plurality of processors may be in operative communication with each other and may be collectively configured to perform one or more functionalities of apparatus 200 as described herein. The plurality of processors may be embodied on a single computing device or distributed across a plurality of computing devices collectively configured to function as scheduling system 104, resource allocation system 106, care coordination apparatus 108, predictive care coordination model 110, care coordination engine 112, and/or apparatus 200. In some example embodiments, the processor 212 may be configured to execute instructions stored in the memory 214 or otherwise accessible to the processor 212. As such, whether configured by hardware or by a combination of hardware and software, the processor 212 may represent an entity (e.g., physically embodied in circuitry—in the form of processing circuitry 210) capable of performing operations according to embodiments of the present disclosure while configured accordingly. Thus, for example, when the processor 212 is embodied as an ASIC, FPGA, or the like, the processor 212 may be specifically configured hardware for conducting the operations described herein. Alternatively, as another example, when the processor 212 is embodied as an executor of software instructions, the instructions may specifically configure the processor 212 to perform one or more operations described herein.

In some example embodiments, the memory 214 may include one or more non-transitory memory devices such as, for example, volatile and/or non-volatile memory that may be either fixed or removable. In this regard, the memory 214 may comprise a non-transitory computer-readable storage medium. It will be appreciated that while the memory 214 is illustrated as a single memory, the memory 214 may comprise a plurality of memories. The plurality of memories may be embodied on a single computing device or may be distributed across a plurality of computing devices. The memory 214 may be configured to store information, data, applications, computer program code, instructions and/or the like for enabling apparatus 200 to carry out various functions in accordance with one or more example embodiments. For example, when apparatus 200 is implemented as care coordination apparatus 108, memory 214 may be configured to store computer program code for performing corresponding functions thereof, as described herein according to example embodiments.

Still further, memory 214 may be configured to store routing tables, that facilitate determining the destination of communications received certain components of FIG. 1. Memory 214 may further include reconciliation tables for tracking communication receives, such as clinical event indication data objects, and reconciling them with predicted care coordination data objects, and/or updating the clinical event indication data objects accordingly. The memory 214 may further comprise a database which may comprise historical scheduling data, historical resource allocation data, historical EHR data, and/or training labels, such as but not limited to acuity levels. Still further, according to certain embodiments, the memory 214 may be modified as described herein, to reformat clinical event indication data objects with additional information received, determined and/or generated according to example embodiments.

The memory 214 may be further configured to buffer input data for processing by the processor 212. Additionally or alternatively, the memory 214 may be configured to store instructions for execution by the processor 212. In some embodiments, the memory 214 may include one or more databases that may store a variety of files, content, or data sets. Among the contents of the memory 214, applications may be stored for execution by the processor 212 to carry out the functionality associated with each respective application. In some cases, the memory 214 may be in communication with one or more of the processor 212, user interface 216, and/or communication interface 218, for passing information among components of apparatus 200.

The optional user interface 216 may be in communication with the processing circuitry 210 to receive an indication of a user input at the user interface 216 and/or to provide an audible, visual, mechanical, or other output to the user. As such, the user interface 216 may include, for example, a keyboard, a mouse, a display, a touch screen display, a microphone, a speaker, and/or other input/output mechanisms. As such, in embodiments in which apparatus 200 is implemented as the scheduling system 104, the user interface 216 may, in some example embodiments, provide means for user entry (such as by a patient or staff member of the healthcare provider) of details pertaining to scheduling of an appointment, and/or the like. The user interface 216, such as when apparatus 200 is implemented as a care coordination apparatus 108, may be further configured to display or provide messages or reports pertaining to predicted care coordination data objects, predicted acuity levels, and/or the like. According to certain embodiments, a user interface 216 may be used to create or generate new scheduling data and/or resource allocation data. For example, an indication to schedule a new appointment may be initiated via user input provided via the user interface 216. A user interface 216 may enable a user to input other types and formats of data. In some example embodiments, such as when apparatus 200 is embodied by a predictive care coordination model 110, aspects of user interface 216 may be limited or the user interface 216 may not be present.

The communication interface 218 may include one or more interface mechanisms for enabling communication with other devices and/or networks. In some cases, the communication interface 218 may be any means such as a device or circuitry embodied in either hardware, or a combination of hardware and software that is configured to receive and/or transmit data from/to a network and/or any other device or module in communication with the processing circuitry 210. By way of example, the communication interface 218 may be configured to enable communication amongst any of the components of FIG. 1, and/or apparatus 200 over a network. Accordingly, the communication interface 218 may, for example, include supporting hardware and/or software for enabling wireless and/or wireline communications via cable, digital subscriber line (DSL), universal serial bus (USB), Ethernet, or other methods.

A network, such as the network in which any of the systems of Figure or components thereof or components described herein may operate, may include a local area network, the Internet, any other form of a network, or any combination thereof, including proprietary private and semi-private networks and public networks. The network may comprise a wired network and/or a wireless network (e.g., a cellular network, wireless local area network, wireless wide area network, some combination thereof, and/or the like).

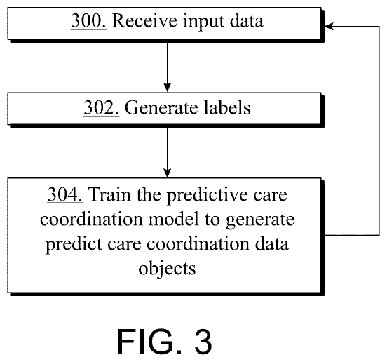

FIG. 3 is a flowchart illustrating example operations of an apparatus 200, such as a care coordination apparatus 108, according to some example embodiments. As shown by operation 300, apparatus 200 may include means, such as processor 212, memory 214, communication interface 218, and/or the like, for receiving input data, such as EHR data from datastore 114, scheduling system 104, and/or the like.

As shown by operation 302, apparatus 200 may include means, such as processor 212, memory 214, communication interface 218, and/or the like, for generating labels. A label may include one or more data points, such as but not limited to data fields comprised by a predicted care coordination data object. A label may include known or true data associated with a clinical event indication data object. For example, a label may include an acuity level as determined by a nurse or other practitioner. A label can include actual appointment times logged by a system or staff member after completion of an appointment. A label can include actual resources, such as supplies, medication, staff, equipment, or the like, noted as utilized for an appointment that has occurred.

According to certain embodiments, a label may be input by a nurse or other practitioner for the purposes of training the predictive care coordination model 110. However, according to certain embodiments, the labels may be generated from data available to the care coordination apparatus 108, such as from an EHR, datastore 114, the scheduling system 104, and/or the resource allocation system 106. As an example, the care coordination apparatus 108 can generate a label based on actual data collected following a patient appoint, such as skilled care duration, appointment duration, and/or the like.

As shown by operation 304, apparatus 200 may include means, such as processor 212, memory 214, communication interface 218, and/or the like, for training the predictive care coordination model 110 to generated predicted care coordination data objects. A machine learning framework and process is applied to the model such that the model determine and applies different weights to different features, such as but not limited to the features and corresponding weights listed in Table 1.

TABLE 1

| Feature | Feature Importance |
|---|---|
| drug_num | 0.323191 |
| is_hypersen | 0.116022 |
| ch_emetic_num | 0.109911 |
| route_num | 0.088045 |
| ch_drug_num | 0.074654 |
| appt_duration_min | 0.064554 |
| has_cancer | 0.026462 |
| is_cid1 | 0.014661 |
| dx_Benign | 0.012487 |
| dx_Rectal | 0.011467 |
| cnt_access_attempts_raw | 0.010148 |
| dx_Multiple Myeloma | 0.008270 |
| is_last_new_low | 0.007188 |
| dx_NHL | 0.007009 |
| cnt_all_problem_raw | 0.006637 |
| dx_Breast | 0.006543 |
| dx_Pancreatic | 0.006431 |
| is_last_plt_low | 0.005656 |
| age | 0.005555 |
| pain_in_6m | 0.005315 |
| is_english | 0.005142 |
| last_plt | 0.004807 |
| last_hgb | 0.004785 |
| is_last_hgb_low | 0.004651 |
| ecog_in_6m | 0.004573 |
| dx_Colon | 0.004573 |
| stage_grp_num | 0.004563 |
| dischargetype_Stretcher | 0.003963 |
| alb_loss_pct_raw | 0.003962 |
| last_albumin | 0.003961 |
| dx_Secondary Cancer Metastasis | 0.003960 |
| hgb_loss_pct_raw | 0.002964 |
| is_last_albuminlow | 0.002963 |
| dischargetype_AmbulatoryHumanAssistance | 0.002963 |
| plt_loss_pct_raw | 0.002962 |
| depressed_in_6m_num | 0.002961 |
| last_neu | 0.002960 |
| neu_loss_pct_raw | 0.002959 |
| dx_NSCLC | 0.002358 |
| dischargetype_Ambulatory | 0.002357 |
| dischargetype_AmbulatoryAssistiveDevice | 0.002356 |
| alb_loss_pct | 0.001264 |
| hgb_loss_pct | 0.001263 |
| plt_loss_pct | 0.001262 |
| dischargetype_Wheelchair | 0.001261 |

As shown by the arrow from 304 to 300, the predictive care coordination model 110 may be iteratively trained and updated, which can account for changes in EHR data, changing trends with respective to clinical care and/or the like.

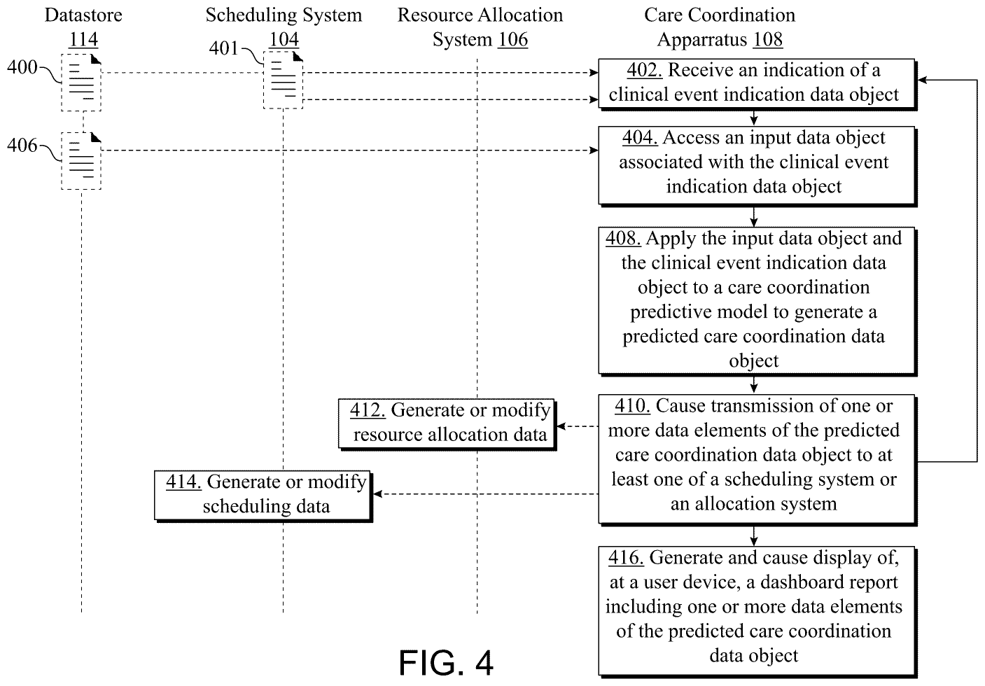

FIG. 4 is a diagram illustrating operations that may be performed, and data that is transmitted, according to certain example embodiments. As shown by operation 304, care coordination apparatus 108, such as apparatus 200, may include means, such as processor 212, memory 214, communication interface 218, and/or the like, for receiving an indication of a clinical event indication data object, such as clinical even indication data object 400 and/or 401 from datastore 114 and/or scheduling system 104, respectively. The clinical event indication data object is representative of a scheduled appointment or appointment requested to be scheduled. The care coordination apparatus 108 may receive a clinical event indication data object in response to being notified by the scheduling system 104. As another example, the care coordination apparatus 108 may access scheduling data from the scheduling system 104 on a routine basis, such as daily.

As shown by operation 404, care coordination apparatus 108, such as apparatus 200, may include means, such as processor 212, memory 214, communication interface 218, and/or the like, for accessing an input data object associated with the clinical event indication data object. According to certain embodiments, the input data object comprises or is generated from an EHR associated with the clinical event indication data object. For example, input data object 406 can include any data from an EHR stored in datastore 114.

As shown by operation 408, care coordination apparatus 108, such as apparatus 200, may include means, such as processor 212, memory 214, and/or the like, for applying the input data object and the clinical event indication data object to a predictive care coordination model 110 to generate a predicted care coordination data object.

As shown by operation 410, care coordination apparatus 108, such as apparatus 200, may include means, such as processor 212, memory 214, communication interface 218, and/or the like, for causing transmission of one or more data elements of the predicted care coordination data object to at least one of a scheduling system or an allocation system. The one or more data elements may be selected from the predicted care coordination data object according to an API of the scheduling system 104 and/or resource allocation system 106. For example, according to certain embodiments, the care coordination apparatus 108 can transmit a predicted appointment duration, or predicted skilled care duration to the scheduling system 104. As another example, the care coordination apparatus 108 can transmit an indication of a staff member type or skillset, such as a nurse.

As shown by operation 412, resource allocation system 106 such as apparatus 200, may include means, such as processor 212, memory 214, communication interface 218, and/or the like, for generating or modifying resource allocation data. In this regard, the received predictive care coordination data object impacts creating of new resource allocation data and/or a change, modification, or update to the resource allocation data. For example, allocation of special equipment, such as an infusion pump, can be allocated for the associated clinical event indication (e.g., scheduled appointment). The generation, modification, or update can occur automatically based on the communication with the care coordination apparatus 108.

As shown by operation 414, scheduling system 104 such as apparatus 200, may include means, such as processor 212, memory 214, communication interface 218, and/or the like, for generating or modifying updating scheduling data. In this regard, a predicted skilled care duration (e.g., nursing time), appointment duration, and/or the like, can be generated, updated, or modified in the scheduling data based on one or more data elements of a predicted care coordination data object. The generation, update or modification can occur automatically based on the communication with the care coordination apparatus 108.

As shown by the arrow from operation 410 returning to 402, the operations may continue to be performed, such as on a routine basis, such as daily, or the like. According to certain embodiments, the iterations may occur automatically as directed by stored computer program code. For example, on a daily basis, or other routine basis, and for each instance of a clinical event indication data object known to a scheduling system 104 and/or communicated to care coordination apparatus 108, the care coordination apparatus 108 may apply it to the care coordination predictive model. Additionally or alternatively, the operations may be processed for scheduled appointments occurring in the next X number of days, such as the next 8 days, to improve the scheduling system and the resource allocation system especially with regard to scheduling and resource allocation over the next X number of days. In this regard, operation 404 may include accessing an updated input data object associated with the clinical event indication data object, and the additional operations of FIG. 4 are repeated accordingly based on the updated input data object. FIG. 4 may further provide for accessing a scheduling system on a routine basis (e.g., daily) to generate updated predicted care coordination data objects, wherein the at least one of the scheduling data or the allocation data is further updated based on the updated input data objects.

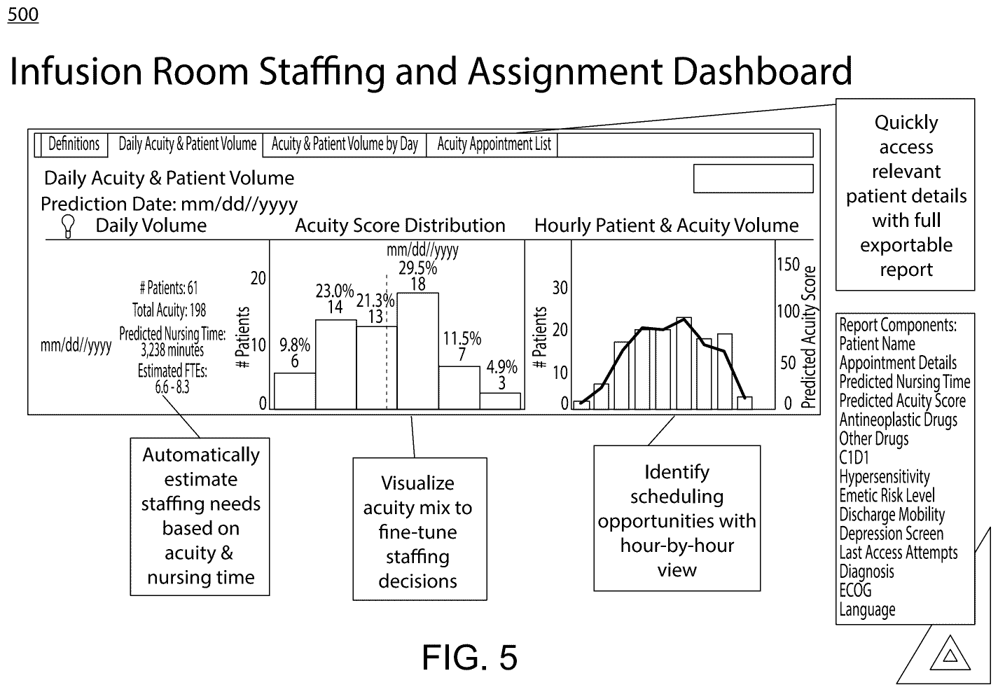

As shown by operation 416, resource allocation system 106 such as apparatus 200, may include means, such as processor 212, memory 214, user interface 216, communication interface 218, and/or the like, for generating and causing display of, at a user device, a dashboard report including one or more data elements of the predicted care coordination data object. The dashboard provided in FIG. 5 is an example of such a dashboard that may be provided and displayed.

FIG. 5 illustrates a user interface 500 that provides a comprehensive dashboard for infusion room staffing and patient acuity management according to certain example embodiments. The dashboard can assist users such as clinical staff, in understanding scheduling and allocation needs related to acuity. The dashboard integrates predicted care coordination data objects generated by the care coordination apparatus, including daily acuity and patient volume, hourly patient and acuity volume, and estimated nursing time. The dashboard includes exportable report components such as patient name, appointment details, predicted nursing time, predicted acuity score, drug information, C1D1 status, hypersensitivity and emetic risk, discharge mobility, depression screening, diagnosis, ECOG score, and language. Certain example embodiments automatically estimates staffing needs based on acuity and nursing time, visualizes acuity mix to fine-tune staffing decisions, identifies scheduling opportunities via hour-by-hour views, and enables rapid access to patient details.

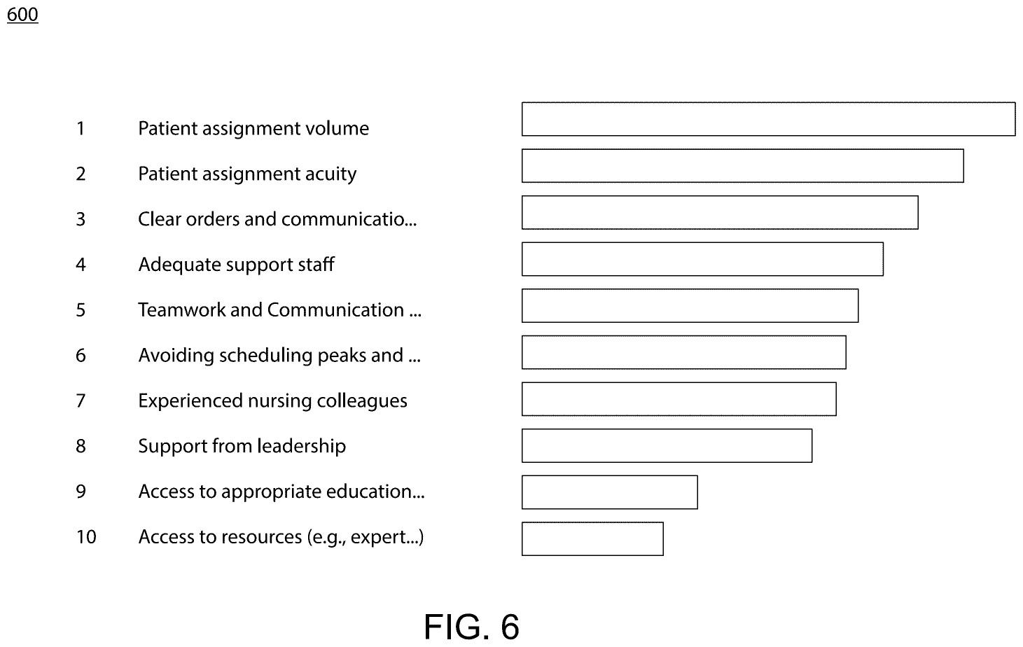

FIG. 6 depicts a user interface 600 that presents qualitative factors influencing staffing decisions, used in conjunction with predicted care coordination data objects according to certain example embodiments. These factors include patient assignment volume and acuity, clarity of orders and communication, availability of support staff, teamwork, scheduling balance, experience of nursing colleagues, leadership support, access to education, and expert resources. User interface 600 complements the predictive model by incorporating human factors into staffing and resource allocation decisions.

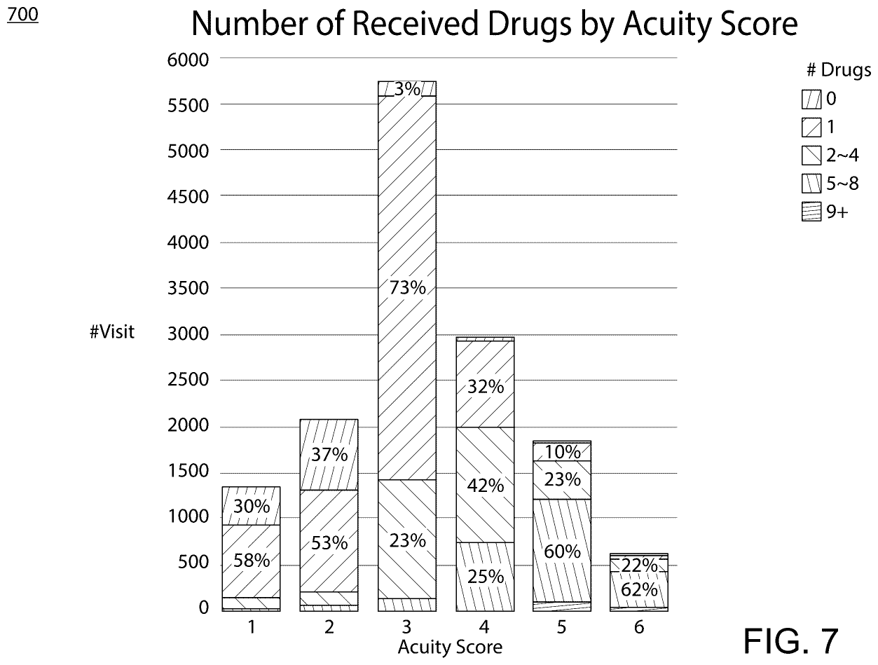

FIG. 7 shows a user interface 700 that visualizes the number of received drugs per patient visit, segmented by predicted acuity score according to certain example embodiments. Certain example embodiments therefore support analysis of drug administration patterns across acuity levels, identification of resource-intensive visits, and integration with the resource allocation system to forecast pharmaceutical needs.

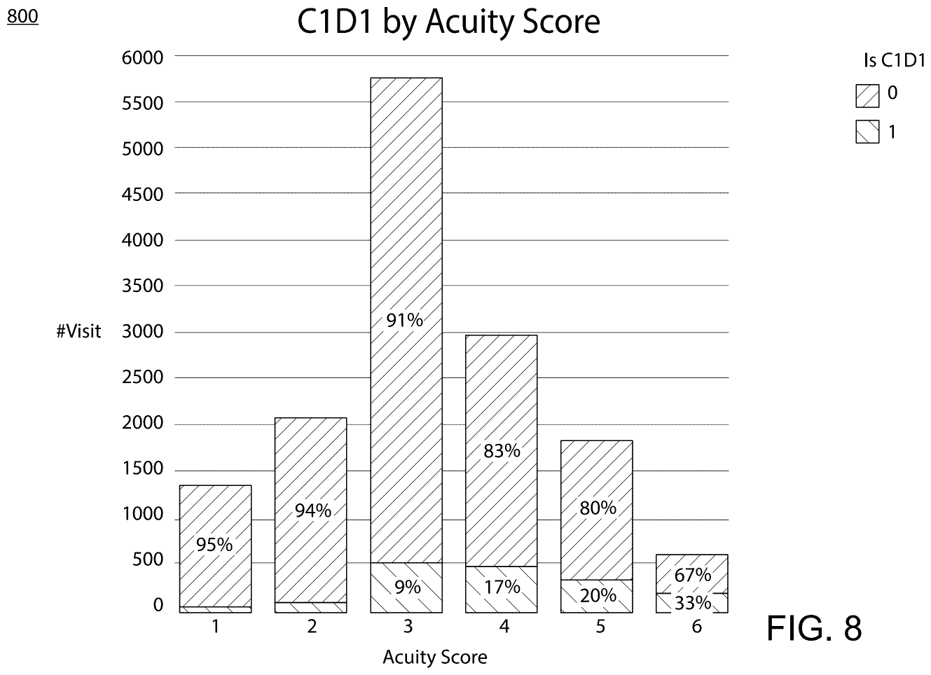

FIG. 8 presents a user interface 800 displaying the proportion of visits involving Cycle 1 Day 1 (C1D1) treatments across acuity scores according to certain example embodiments. Certain example embodiments therefore support prediction of initial treatment complexity, adjustment of skilled care duration and staffing via the scheduling system, and enhanced acuity modeling for new treatment starts.

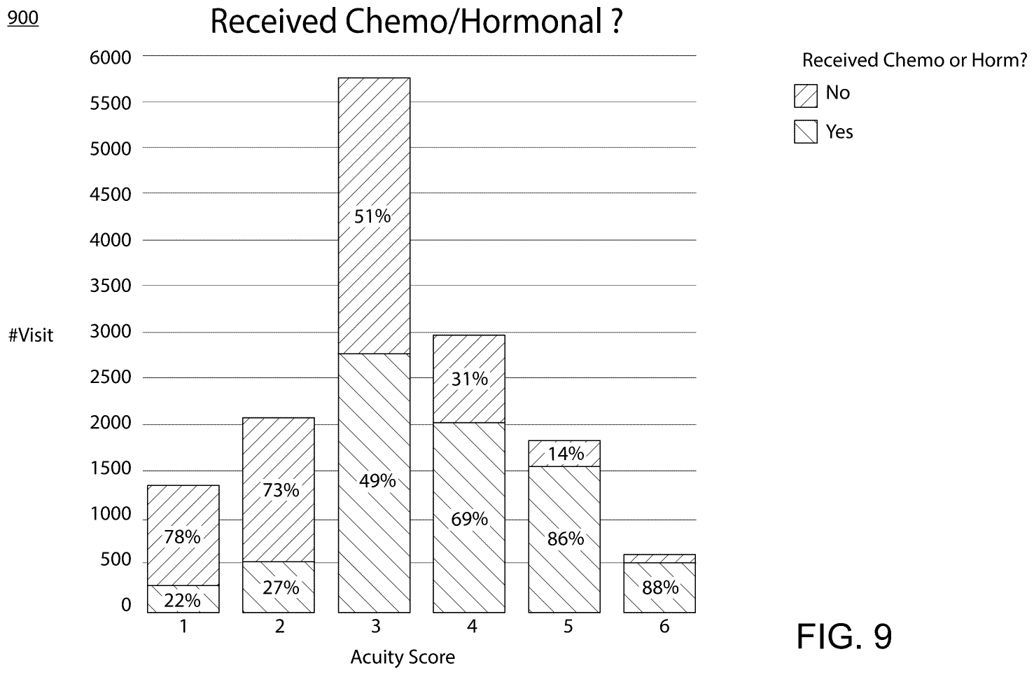

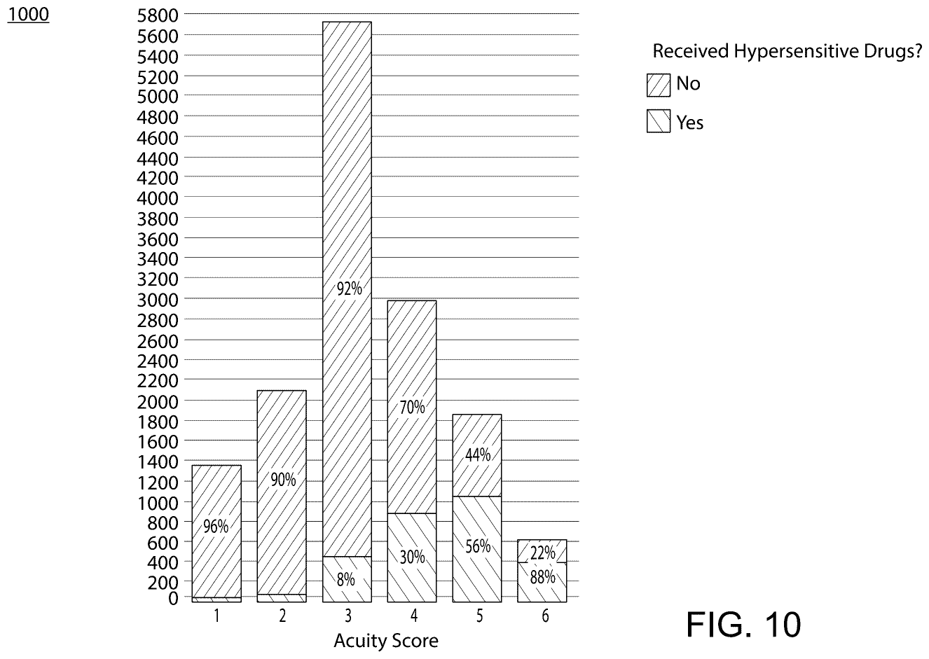

FIG. 9 illustrates a user interface 900 showing the distribution of patients receiving chemotherapy or hormonal therapy across acuity scores according to certain example embodiments. Certain example embodiments therefore enables correlation of treatment type with acuity, improved prediction of nursing time and supply needs, and integration with the datastore for training the predictive model.

FIG. 10 displays a user interface 1000 visualizing the frequency of hypersensitivity drug administration across acuity scores according to certain example embodiments. Certain example embodiments therefore support identification of high-risk appointments, adjustment of staffing skill level and monitoring protocols, and real-time updates to scheduling and allocation systems based on changes in electronic health record data.

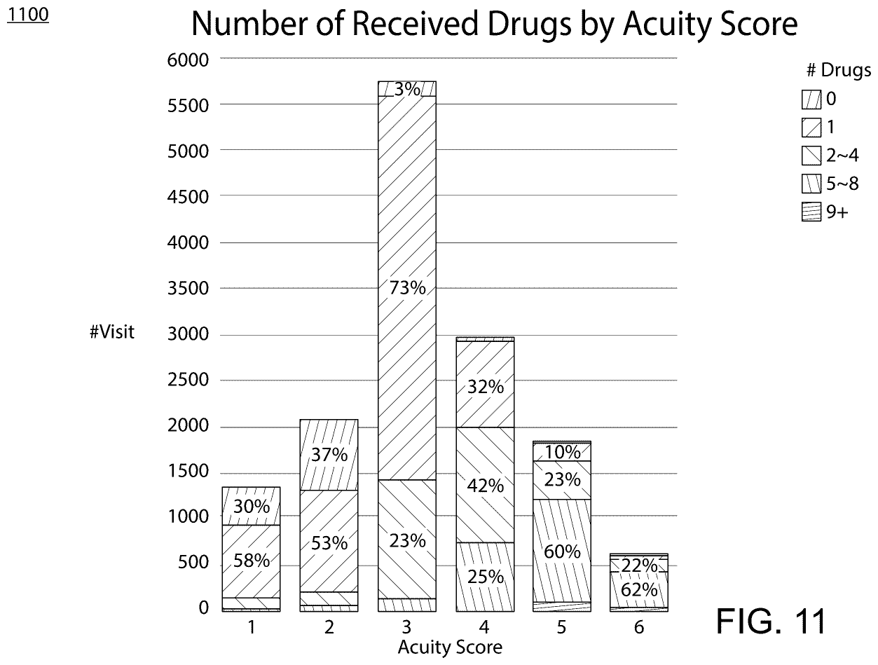

FIG. 11 provides a user interface 1100 visualizing drug volume per visit by acuity score, reinforcing insights from the previous drug volume interface according to certain example embodiments. It may include enhanced filtering or sorting capabilities and integration with dashboard reports generated by the care coordination engine.

Example embodiments disclosed herein provide improvements to the technological field of healthcare scheduling and resource allocation. These improvements are realized through a system architecture that integrates a care coordination apparatus with a predictive care coordination model, enabling dynamic, real-time, and context-sensitive updates to scheduling and resource allocation systems. The following scenario illustrates the technical advantages of the system.

On day 1, Suzy is scheduled for an outpatient oncology infusion appointment to occur on day 10. The scheduling system 104 initially assigns a default skilled care duration and appointment duration based on available EHR data. The care coordination apparatus 108 applies this data to the predictive care coordination model 110, generating a predicted care coordination data object that reflects a skilled care duration of 1 hour and an appointment duration of 1.5 hours.

On day 7, Suzy undergoes additional healthcare interactions—such as lab tests, imaging studies, or prescription changes—that result in updates to her EHR. These updates are automatically detected by the care coordination engine 112, which re-applies the updated input data object to the predictive model. The model generates a new predicted care coordination data object, now reflecting a skilled care duration of 1.5 hours and an appointment duration of 2 hours. These updated predictions are automatically transmitted to the scheduling system 104 and resource allocation system 106, which adjust staffing, equipment, and appointment availability accordingly and without requiring manual intervention.

The predictive care coordination model 110 is implemented within a machine learning framework that provides technical capabilities beyond what can be achieved by human reasoning or conventional rule-based systems. Unlike traditional models that rely on static feature sets or manually curated rules, the predictive model disclosed herein dynamically ingests and interprets vast, heterogeneous, and evolving datasets from electronic health records (EHRs). These datasets may include thousands of structured and unstructured data points per patient, encompassing historical medical visits, diagnostic codes, lab results, medication regimens, imaging studies, and more.

Certain example embodiments are designed to operate in a domain where the dimensionality and variability of data exceed human cognitive limits. For example, the model can process and learn from tens of thousands of unique prescription drug types, each with varying interactions, emetic risks, and hypersensitivity profiles. Certain example embodiments continuously adapt to new clinical findings, emerging treatment protocols, and evolving diagnostic standards, such as newly introduced lab tests or updated oncology staging criteria, without requiring manual reprogramming.

Moreover, the predictive care coordination model a purpose-built, domain-specific model trained using labeled data objects that reflect real-world care coordination outcomes, such as actual skilled care durations, acuity levels, and resource utilization. These labels are generated and refined through integration with the scheduling system 104, resource allocation system 106, and datastore 114, enabling continuous learning and model evolution.

Certain example embodiments automatically generate, update, and transmit predicted care coordination data objects in response to real-time changes in EHR data, thereby enabling a fundamental shift in how healthcare systems operate. Scheduling and resource allocation is therefore transformed from static, reactive processes into dynamic, predictive, and adaptive systems such that the scheduling system and resource allocation system is improved accordingly. Certain example embodiments therefore provide an intelligent infrastructure that cannot be replicated by human effort or by conventional machine learning models lacking the architectural integration and domain-specific training described herein.

By integrating a care coordination apparatus with a predictive care coordination model trained on EHR data, the system enables automated, forward-looking estimation of scheduling and resource allocation. For example, predictions can be generated for the current day and up to seven days in advance, allowing dynamic adjustment of scheduling data and resource allocation data based on anticipated acuity levels and skilled care durations. Additionally, certain example embodiments continuously update predictions based on new EHR inputs, ensuring accurate and adaptive staffing decisions. By automatically interfacing with both the scheduling system and the resource allocation system, the predictive care coordination model disclosed herein significantly reduces the need for manual intervention traditionally required to respond to staffing shortages, resource constraints, and fluctuating patient acuity.

Conventional systems often rely on practitioners to detect and react to scheduling and resource allocation deficiencies or issues, which often may occur too late and have a negative impact to scheduling and resource distribution within an organization, and negative impact to the scheduling system and resource allocation system. Such reaction-based interactions with conventional systems depend on practitioners to access user interfaces of both the scheduling system and resource allocation system, to react and adjust scheduling and resource allocation accordingly to attempt to satisfy patient needs. The interactions required by conventional systems result in delayed responses, inefficient resource deployment, and increased operational overhead. In contrast, certain example embodiments continuously monitor electronic health record (EHR) data and dynamically generate predicted care coordination data objects that proactively inform and update scheduling and allocation decisions. This automation not only improves accuracy and responsiveness but also reduces the computational and human resource burden on the underlying systems. As a result, the scheduling system and resource allocation system operate more efficiently, consuming fewer resources while maintaining or improving care quality, staff satisfaction, and operational throughput. These improvements are not achievable through manual workflows or conventional machine learning models lacking the integrated architecture and domain-specific training described herein.

It will be appreciated that the figures are each provided as examples and should not be construed to narrow the scope or spirit of the disclosure in any way. In this regard, the scope of the disclosure encompasses many potential embodiments in addition to those illustrated and described herein. Numerous other configurations may also be used to implement embodiments of the present disclosure. For example, in addition to or alternatively to utilizing example embodiments with a clinical oncology environment, example embodiments disclosed herein may be applied to healthcare providers that provide other healthcare services, such as but not limited to radiological surgery, nurse navigation, or the like.

FIGS. 4 and 5 illustrate operations of a method, apparatus, and computer program product according to some example embodiments. It will be understood that each operation of the flowcharts or diagrams, and combinations of operations in the flowchart or diagrams, may be implemented by various means, such as hardware and/or a computer program product comprising one or more computer-readable mediums having computer readable program instructions stored thereon. For example, one or more of the procedures described herein may be embodied by computer program instructions of a computer program product. In this regard, the computer program product(s) which embody the procedures described herein may comprise one or more memory devices of a computing device (for example, memory 214) storing instructions executable by a processor in the computing device (for example, by processor 212). In some example embodiments, the computer program instructions of the computer program product(s) which embody the procedures described above may be stored by memory devices of a plurality of computing devices. As will be appreciated, any such computer program product may be loaded onto a computer or other programmable apparatus (for example, apparatus 200) to produce a machine, such that the computer program product including the instructions which execute on the computer or other programmable apparatus creates means for implementing the functions specified in the flowchart block(s). Further, the computer program product may comprise one or more computer-readable memories on which the computer program instructions may be stored such that the one or more computer-readable memories can direct a computer or other programmable apparatus to function in a particular manner, such that the computer program product may comprise an article of manufacture which implements the function specified in the flowchart block(s). The computer program instructions of one or more computer program products may also be loaded onto a computer or other programmable apparatus (for example, apparatus 200 and/or other apparatus) to cause a series of operations to be performed on the computer or other programmable apparatus to produce a computer-implemented process such that the instructions which execute on the computer or other programmable apparatus implement the functions specified in the flowchart block(s).

Accordingly, blocks of the flowchart support combinations of means for performing the specified functions and combinations of operations for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

Many modifications and other embodiments of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. A system comprising a memory and one or more processors communicatively coupled to the memory, the one or more processors configured to:
receive an indication of a clinical event indication data object;
access an input data object associated with the clinical event indication data object;
apply the input data object and the clinical event indication data object to a care coordination predictive model to generate a predicted care coordination data object; and
cause transmission of one or more data elements of the predicted care coordination data object to at least one of a scheduling system or an allocation system, wherein at least one of scheduling data associated with the clinical event indication data object or allocation data associated with the clinical event indication data object is determined or modified is based on the predicted care coordination data object.

2. The system according to claim 1, wherein the memory and the one or more processors are further configured to:
receive an indication of a change to the input data object associated with the clinical event indication data object;
in response to receiving the indication of the change, apply the input data object and the clinical event indication data object to generate an updated predicted care coordination data object; and
cause transmission of one or more data elements of the predicted care coordination data object to at least one of a scheduling system or an allocation system, wherein at least one of scheduling data associated with the clinical event indication data object or allocation data associated with the clinical event indication data object is modified based on the predicted care coordination data object.

3. The system according to claim 1, wherein the input data object comprises or is generated from an electronic health record (EHR) associated with the clinical event indication data object.

4. The system according to claim 1, wherein the memory and the one or more processors are further configured to:
generate and cause display of, at a user device, a dashboard report including one or more data elements of the predicted care coordination data object to at least one of a scheduling system or an allocation system.

5. The system according to claim 1, wherein the one or more data elements of the predicted care coordination data object comprises a predicted skilled care duration.

6. The system according to claim 1, wherein the one or more data elements of the predicted care coordination data object comprises an acuity level.

7. The system according to claim 1, wherein the memory and the one or more processors are further configured to:
access an updated input data object associated with the clinical event indication data object; and
apply the updated input data object to the care coordination predictive model to generate an updated predicted care coordination data object, wherein the at least one of the scheduling data or the allocation data is further updated based on the updated input data object.

8. The system according to claim 1, wherein the memory and the one or more processors are further configured to:
access a scheduling system on a routine basis to generate updated predicted care coordination data objects, wherein the at least one of the scheduling data or the allocation data is further modified based on the updated predicted care coordination data objects.

9. A computer-implemented method comprising:
receiving an indication of a clinical event indication data object;
accessing an input data object associated with the clinical event indication data object;
applying the input data object and the clinical event indication data object to a care coordination predictive model to generate a predicted care coordination data object; and
causing transmission of one or more data elements of the predicted care coordination data object to at least one of a scheduling system or an allocation system, wherein at least one of scheduling data associated with the clinical event indication data object or allocation data associated with the clinical event indication data object is determined or modified is based on the predicted care coordination data object.

10. The computer-implemented method according to claim 9, further comprising:
receiving an indication of a change to the input data object associated with the clinical event indication data object;
in response to receiving the indication of the change, applying the input data object and the clinical event indication data object to generate an updated predicted care coordination data object; and
causing transmission of one or more data elements of the predicted care coordination data object to at least one of a scheduling system or an allocation system, wherein at least one of scheduling data associated with the clinical event indication data object or allocation data associated with the clinical event indication data object is modified based on the predicted care coordination data object.

11. The computer-implemented method according to claim 9, wherein the input data object comprises or is generated from an electronic health record (EHR) associated with the clinical event indication data object.

12. The computer-implemented method according to claim 9, further comprising:
generating and causing display of, at a user device, a dashboard report including one or more data elements of the predicted care coordination data object to at least one of a scheduling system or an allocation system.

13. The computer-implemented method according to claim 9, wherein the one or more data elements of the predicted care coordination data object comprises a predicted skilled care duration.

14. The computer-implemented method according to claim 9, wherein the one or more data elements of the predicted care coordination data object comprises an acuity level.

15. The computer-implemented method according to claim 9, further comprising:
access an updated input data object associated with the clinical event indication data object; and
apply the updated input data object to the care coordination predictive model to generate an updated predicted care coordination data object, wherein the at least one of the scheduling data or the allocation data is further updated based on the updated input data object.

16. The computer-implemented method according to claim 9, further comprising:
access a scheduling system on a routine basis to generate updated predicted care coordination data objects, wherein the at least one of the scheduling data or the allocation data is further modified based on the updated predicted care coordination data objects.

17. A computer program product comprising at least one non-transitory computer-readable storage medium having computer-executable program code instructions stored therein, the computer-executable program code instructions comprising program code instructions to:
receive an indication of a clinical event indication data object;
access an input data object associated with the clinical event indication data object;
apply the input data object and the clinical event indication data object to a care coordination predictive model to generate a predicted care coordination data object; and
cause transmission of one or more data elements of the predicted care coordination data object to at least one of a scheduling system or an allocation system, wherein at least one of scheduling data associated with the clinical event indication data object or allocation data associated with the clinical event indication data object is determined or modified is based on the predicted care coordination data object.

18. The computer program product according to claim 17, wherein the computer-executable program code instructions comprise program code instructions to:
receive an indication of a change to the input data object associated with the clinical event indication data object;
in response to receiving the indication of the change, apply the input data object and the clinical event indication data object to generate an updated predicted care coordination data object; and
cause transmission of one or more data elements of the predicted care coordination data object to at least one of a scheduling system or an allocation system, wherein at least one of scheduling data associated with the clinical event indication data object or allocation data associated with the clinical event indication data object is modified based on the predicted care coordination data object.

19. The computer program product according to claim 17, wherein the input data object comprises or is generated from an electronic health record (EHR) associated with the clinical event indication data object.

20. The computer program product according to claim 17, wherein the computer-executable program code instructions comprise program code instructions to:
generate and cause display of, at a user device, a dashboard report including one or more data elements of the predicted care coordination data object to at least one of a scheduling system or an allocation system.
